(19) United States
(12) Patent Application Publication
(10) Pub. No.: US 2005/0084938 A1
(43) Pub. Date: Apr. 21, 2005

(54) Method for plasmid preparation by conversion of open circular plasmid to supercoiled plasmid

(76) Inventor: Edward D. Hyman, Metairie, LA (US)

Correspondence Address: NIXON & VANDERHYE, PC, 1100 N GLEBE ROAD, 8TH FLOOR, ARLINGTON, VA, 22201-4714, US

(21) Appl. No.: 10/837,684

(22) Filed: May 4, 2004

Related U.S. Application Data
(63) Continuation-in-part of application No. 10/799,638, filed on Mar. 15, 2004.
(60) Provisional application No. 60/560,749, filed on Mar. 25, 2003.
(60) Provisional application No. 60/608,923, filed on Jul. 2, 2003.
(60) Provisional application No. 60/541,941, filed on Feb. 6, 2004.

(51) Int. Cl. C12Q 1/68; C12P 19/34; C12N 15/85

(52) U.S. Cl. 435/91.2; 435/455

(57) ABSTRACT

In one embodiment of the invention, a method is provided for preparing plasmid from host cells which contain the plasmid, comprising: (a) providing a plasmid solution comprised of unligatable open circular plasmid; (b) reacting the unligatable open circular plasmid with one or more enzymes and appropriate nucleotide cofactors, such that unligatable open circular plasmid is converted to 3′-hydroxyl, 5′-phosphate nicked plasmid; (c) reacting the 3′-hydroxyl, 5′-phosphate nicked plasmid with a DNA ligase and DNA ligase nucleotide cofactor, such that 3′-hydroxyl, 5′-phosphate nicked plasmid is converted to relaxed covalently closed circular plasmid; and (d) reacting the relaxed covalently closed circular plasmid with a DNA gyrase and DNA gyrase nucleotide cofactor, such that relaxed covalently closed circular plasmid is converted to negatively supercoiled plasmid. In other embodiments, DNA gyrase is replaced by reverse DNA gyrase or reaction (d) is not performed.

### CROSS-REFERENCE TO RELATED APPLICATIONS

This application is a continuation-in-part of U.S. patent application Ser. No. 10/799,638, filed Mar. 15, 2004; which claims the benefit of provisional U.S. Appln. No. 60/541,941, filed Feb. 6, 2004; provisional U.S. application Ser. No. 10/612,636, filed Jul. 2, 2003; and provisional U.S. application Ser. No. 10/396,880, filed Mar. 25, 2003; the contents of which are incorporated by reference herein.

### BACKGROUND OF THE INVENTION

Plasmids are double stranded, circular, extrachromosomal DNA molecules (plasmids are defined as such herein). Plasmids are contained inside host cells. One common host cell is Escherichia coli (E. coli). Many other types of cells are known to carry plasmids. This includes other bacteria, yeast, and higher eukaryotic cells. Plasmids may be artificial (i.e., manmade), such as cloning vectors carrying foreign DNA inserts. Plasmids may also occur naturally, such as in mitochondria and chloroplasts.

Since the invention of cloning circa 1975, the preparation of plasmid has been a routine task in molecular biology. In the ensuing time, plasmid preparation has become a highly crowded art. The crowded nature of the art is a reflection of the widespread importance of the procedure in molecular biology. Numerous articles and patents have been published in the past 25 years describing novel methods for preparing plasmid. The problem of plasmid preparation has attracted enormous commercial interest. Companies sell kits for plasmid preparation (Amersham, Qbiogene, Clonetech, Promega, Biorad, Qiagen, Sigma); proprietary resins for purifying plasmid (Qiagen, Amersham, Puresyn, Macherey-Nagel); and automated instruments for preparing plasmid (Qiagen, MacConnell, Autogen).

In the purification of plasmid from host cells, the final plasmid preparation is usually a mixture of two main forms of plasmid: open circular and supercoiled. In the supercoiled form, the plasmid has a covalently closed circular form, and the plasmid is negatively supercoiled in the host cell by the action of host enzymes. In the open circular form, one strand of the DNA duplex is broken at one or more places. The single strand break(s) in an open circular plasmid results in a relaxed topology.

Open circular plasmid in a plasmid preparation can result from several causes. Open circular plasmid may exist in the host cells immediately prior to lysis. Some supercoiled plasmid in the host cells may unintentionally be converted to open circular plasmid in the preparation of a cleared lysate (e.g. by chemical hydrolysis and/or physical shearing) due to the fragile nature of supercoiled plasmid. Additional plasmid purification procedures, such as organic extraction, precipitation, ultrafiltration, and chromatography, may unintentionally convert some supercoiled plasmid from the cleared lysate to open circular plasmid, due to the fragile nature of supercoiled plasmid.

For most plasmid applications, the active plasmid form is supercoiled. Open circular plasmid is often either inactive or poorly active. Plasmid for gene transfer (e.g. in vitro DNA transformation or in vivo DNA therapy) may require a high percentage of supercoiled plasmid and a low percentage of open circular plasmid contamination. Numerous methods have been described in the prior art to achieve this objective.

Le Brun et al. described a method for purifying supercoiled plasmid from open circular plasmid using agarose gel electrophoresis (BioTechniques 6:836-838, 1988). Separation was based on differential migration in agarose gel. Supercoiled plasmid was recovered from the ethidium bromide stained gel. Hediger described a similar method using continuous elution (Anal. Biochem. 159:280-286, 1986).

Gorich et al. described a method for purifying supercoiled plasmid from open circular plasmid using polyacrylamide gel electrophoresis (Electrophoresis 19:1575-1576, 1998). Separation was based on differential migration in polyacrylamide gel. Supercoiled plasmid was recovered from the gel by electrophoretic elution.

Womble et al. described a method for purifying supercoiled plasmid using density gradient centrifugation (J. Bacteriol. 130:148-153, 1977). Plasmid was dissolved in a cesium chloride-ethidium bromide solution and centrifuged at high speed. Supercoiled plasmid was separated from open circular plasmid based on differential incorporation of ethidium bromide.

Best et al. described a method for purifying supercoiled plasmid using reverse phase chromatography (Anal. Biochem. 114:235-243, 1981). The chromatographic resin separated supercoiled from open circular forms. Many chromatographic methods have been described in the prior art for separating supercoiled plasmid from open circular plasmid. This includes reverse phase, anion exchange, size exclusion, membrane, and thiophilic chromatography. Several chromatographic resins are commercially available for separating supercoiled from open circular forms (Puresyn, Amersham, Prometic).

Hyman described a method for purifying supercoiled plasmid using selective exonuclease digestion (BioTechniques, 13:550-554, 1992). A cell lysate was incubated with a mixture of exonuclease I and exonuclease III. The exonucleases selectively degraded open circular plasmid and chromosomal DNA without degrading supercoiled plasmid, thereby purifying supercoiled plasmid.

Prior art methods for purifying supercoiled plasmid from open circular plasmid involve separation and removal of open circular plasmid from supercoiled plasmid, or selective degradation of the open circular plasmid. In the chromatographic, electrophoretic, and ultracentrifugation prior art methods for purifying supercoiled plasmid, the open circular plasmid is separated and removed. In the enzymatic prior art methods, open circular plasmid is selectively degraded by exonuclease. One disadvantage of prior art approaches is that the final yield of supercoiled plasmid is reduced because open circular plasmid is removed or degraded. For example, large scale plasmid preparations may contain 10% to 30% open circular plasmid. Thus, using prior art methods, at least 10% to 30% of the total plasmid will be lost during purification of supercoiled plasmid.

The invention overcomes the inherent disadvantage of prior art methods by using a fundamentally different operating principle, by converting open circular plasmid to supercoiled plasmid.

(1) In DNA repair, the enzymatic repair of single stranded breaks in double stranded DNA is known. Laipis et al. used DNA polymerase I and DNA ligase to repair single stranded breaks (Proc. Natl. Acad. Sci. USA 69:3211-3214, 1972). Mitzel-Landbeck et al. used exonuclease III, DNA polymerase I, and DNA ligase to repair single stranded breaks (Biochem. Biophys. Acta 432:145-153, 1976).

(2) In DNA replication, Gellert discovered that the conversion of covalently closed circular plasmid to supercoiled plasmid is accomplished by DNA gyrase (Proc. Natl. Acad. Sci. USA 73:3872-3876, 1976).

(3) In DNA replication, Shlomai et al. converted open circular plasmid, generated from single stranded circular DNA, to double stranded supercoiled plasmid using an enzyme mixture comprising DNA polymerase I, DNA ligase, and DNA gyrase (J. Biol. Chem. 256:5233-5238, 1981).

The prior art neither teaches nor suggests combining these reactions to convert open circular plasmid to supercoiled plasmid. This invention provides an improved method for plasmid preparation.

### SUMMARY OF THE INVENTION

An objective of the invention is to provide a method for preparing supercoiled plasmid, by converting open circular plasmid into supercoiled plasmid enzymatically, thereby achieving a final plasmid preparation which has an increased proportion of supercoiled plasmid. This advantage was not previously appreciated in the prior art.

In one embodiment of the invention, a method is provided for preparing plasmid from host cells which contain the plasmid, comprising: (a) providing a plasmid solution comprised of unligatable open circular plasmid; (b) reacting the unligatable open circular plasmid with one or more enzymes and appropriate nucleotide cofactors, such that at least some unligatable open circular plasmid is converted to 3′-hydroxyl, 5′-phosphate nicked plasmid; (c) reacting the 3′-hydroxyl, 5′-phosphate nicked plasmid with a DNA ligase and DNA ligase nucleotide cofactor, such that at least some 3′-hydroxyl, 5′-phosphate nicked plasmid is converted to relaxed covalently closed circular plasmid; and (d) reacting the relaxed covalently closed circular plasmid with a DNA gyrase and DNA gyrase nucleotide cofactor, such that at least some relaxed covalently closed circular plasmid is converted to negatively supercoiled plasmid. In other embodiments, DNA gyrase is replaced by reverse DNA gyrase or reaction (d) is not performed. Incubations may also include salt, buffer, and nucleotide cofactor appropriate for the enzyme. Reaction conditions such as concentration of the aforementioned chemicals, temperature, and time may be adjusted to provide suitable conversion kinetics and yield.

Preferably, reactions (b), (c), and (d) are performed in a single reaction using an enzyme mixture comprising a DNA polymerase, DNA ligase, and DNA gyrase. Preferably, the mixture further comprises a 3′ deblocking enzyme, such as exonuclease III, AP endonuclease, or 3′-phosphatase. Preferably, the mixture further comprises one or more kinase enzymes and a high energy phosphate donor, which converts nucleotide by-product of the nucleotide cofactor generated by an enzyme in the mixture back to nucleotide cofactor. Preferably, the enzyme mixture further comprises one or more exonucleases, such as exonuclease V, which degrades linear chromosomal DNA without substantially degrading open circular or supercoiled plasmid.

Further embodiments of the invention include kits and compositions comprising one or more of the aforementioned enzymes and optional reaction components (e.g. salt, buffer, nucleotide cofactor). In a kit, enzymes in one or more containers (separate enzyme compositions or a mixture thereof) may be packaged for single or multiple reactions. Instructions (e.g. a printed sheet included in the kit or a label applied to the outside of the kit) for practicing a method of the invention are another optional component of the kit.

Further objectives and advantages will become apparent from a consideration of the ensuing description.

### BRIEF DESCRIPTION OF THE DRAWING

FIG. 1 illustrates an embodiment of the invention.

### DESCRIPTION OF SPECIFIC EMBODIMENTS OF THE INVENTION

In the invention, open circular plasmid is enzymatically converted to supercoiled plasmid. This is accomplished by incubating the open circular plasmid with enzymes, either sequentially or preferably simultaneously with an enzyme mixture. The result of this enzymatic incubation is a plasmid preparation with a higher percentage of supercoiled plasmid and a lower percentage of open circular plasmid. The invention operates in a fundamentally different manner from the prior art. In some embodiments, open circular plasmid is not separated from supercoiled plasmid and is not degraded.

### Preparing the Cleared Lysate

The enzymatic conversion reactions of the invention are preferably performed after obtaining a cleared lysate of host cells containing the plasmid. A “cleared lysate” is a well known term in the art and refers to an aqueous solution containing plasmid, and usually RNA, usually soluble proteins, and usually residual amounts of chromosomal DNA, which is obtained after lysis of host cells and the separation of the cell debris, usually by filtration or centrifugation. Any method for preparing a cleared lysate may be potentially useful. Plasmid in the cleared lysate is usually a mixture of supercoiled and open circular plasmid.

The host cells containing plasmid are preferably bacteria, preferably Escherichia coli. Two methods are commonly used in the art for producing a cleared lysate from bacteria. Both methods comprise lysing the host cells, precipitating chromosomal DNA, and removing the precipitated chromosomal DNA and cell debris. In the alkaline lysis method (Birnboim, Nucl. Acids Res. 7:1513-1523, 1975), host cells are lysed using an alkaline detergent solution. Chromosomal DNA is precipitated by neutralizing the lysed cell solution. The precipitated chromosomal DNA and cell debris is removed by filtration or centrifugation. In the boiling method (Holmes, Anal. Biochem. 114:193-197, 1981), host cells are lysed using lysozyme. Chromosomal DNA is precipitated by heating the lysed cell solution; the precipitated chromosomal DNA and cell debris is removed by centrifugation. Other non-limiting methods of potential use for preparing a cleared lysate may include mechanical disruption methods (U.S. Pat. No. 6,455,287). A preferred method for preparing a cleared lysate is the alkaline lysis method.

After preparing the cleared lysate, the plasmid in the cleared lysate is optionally further purified in any desired manner prior to the enzymatic conversion reactions. Further purification can be accomplished by many methods, such as organic solvent extraction, precipitation, RNA digestion by a ribonuclease, chromatography, electrophoresis, ultrafiltration (such as tangential flow filtration), or combinations thereof. Preferably, the further purification procedure(s) do not separate open circular plasmid from supercoiled plasmid or degrade open circular plasmid. Further purification may be advantageous in several ways. First, further purification may result in plasmid in a buffer which is better suited for the enzymatic conversion reactions. Second, further purification may allow more efficient and reliable enzymatic conversion reactions by removing contaminants (such as protein and RNA) which might inhibit the enzymatic conversion reactions.

After preparing a cleared lysate, and optionally further purifying the plasmid from other host cell components, the resulting plasmid solution comprises open circular plasmid, and usually supercoiled plasmid (i.e., usually a mixture of open circular and supercoiled plasmids).

### Enzymatic Conversion Reactions

The inventor has discovered that the vast majority of open circular plasmid in plasmid preparations is unligatable (defined as open circular plasmid which is not 3′-hydroxyl, 5′-phosphate nicked plasmid), which cannot be converted to relaxed covalently closed circular form using only DNA ligase. Further, the inventor suspects that the percentage of open circular plasmid which is unligatable is higher in plasmid preparations with a large proportion of open circular plasmid. Thus, for plasmid preparations with 30% open circular plasmid, very little open circular plasmid is 3′-hydroxyl, 5′-phosphate nicked plasmid. This is an unexpected and surprising observation, as the prior art would predict that about half of the open circular plasmid would be 3′-hydroxyl, 5′-phosphate nicked plasmid. However, this is not observed experimentally for open circular plasmid in plasmid preparations.

Unintentional DNA damage suffered during plasmid preparation might produce mostly unligatable open circular plasmid. Such unintentional DNA damage is usually unavoidable during preparation of the cleared lysate and subsequent purification procedures due to the fragile nature of supercoiled plasmid.

The unligatable open circular plasmid might be a result of a plurality of different types of DNA damage. For example, some unligatable open circular plasmid may be nicked or may be gapped plasmid. Some unligatable open circular plasmid may have ordinary hydroxyl or phosphate groups at the 3′ and 5′ termini. Alternatively, some unligatable open circular plasmid may have other functional groups at the terminal ends. For example, unintentional free radical damage during plasmid preparation may produce single strand breaks with non-ordinary termini, such as 3′-phosphoglycolate or 5′-aldehyde.

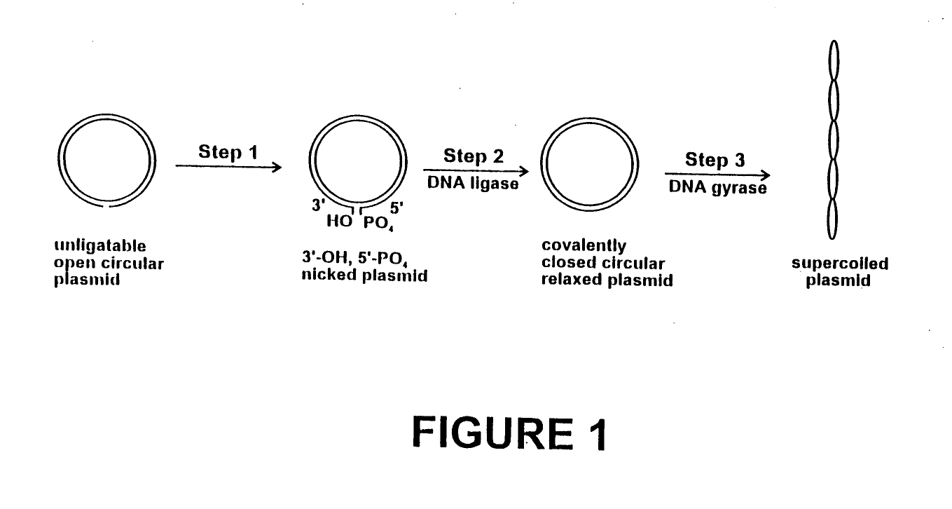

Enzymatic conversion reactions are preferably performed on open circular plasmid in an aqueous solution. One embodiment of the invention preferably comprises at least three enzymatic conversion reactions, illustrated in FIG. 1, which convert unligatable open circular plasmid to supercoiled plasmid. They may be performed sequentially or simultaneously.

First Enzymatic Reaction: Conversion of Unligatable Open Circular Plasmid to 3′-hydroxyl, 5′-phosphate Nicked Plasmid.

In the first enzymatic conversion reaction (first reaction), unligatable open circular plasmid in a plasmid solution is converted in vitro to 3′-hydroxyl, 5′-phosphate nicked plasmid (ligatable form). This can be accomplished in many ways using several different enzymes.

Preferred Mode: In a preferred conversion method, the unligatable open circular plasmid is converted to 3′-hydroxyl, 5′-phosphate nicked plasmid by incubation with a DNA polymerase in the presence of deoxyribonucleoside triphosphate substrates (dNTPs).

A preferred polymerase is DNA polymerase I, which preferably has both 3′-5′ and 5′-3′ exonuclease activities. The 5′-3′ exonuclease activity of DNA polymerase I may advantageously convert some 5′ termini that lack a 5′-phosphate to a 5′-phosphate terminus. This activity is also known as nick translation. The 3′-5′ exonuclease activity of DNA polymerase I may advantageously convert some 3′ termini that lack a 3′-hydroxyl to a 3′-hydroxyl. Example 1 demonstrates non-limiting embodiments of the preferred mode. Other DNA polymerases may be used.

A 3′ deblocking enzyme may optionally be used to assist in the first reaction. Some unligatable open circular plasmid may have a blocking group at the 3′ terminus. The blocking group may inhibit (completely or partially) the ability of DNA polymerase to extend the 3′ terminus. In this case, a 3′ deblocking enzyme can remove the 3′ blocking group and produce a 3′-hydroxyl terminus. The resulting 3′-hydroxyl terminus may then be extended by DNA polymerase. Incubations with 3′ deblocking enzyme and DNA polymerase are preferably performed simultaneously, but could also be performed sequentially in the order 3′ deblocking enzyme followed by DNA polymerase. Non-limiting examples of 3′ deblocking enzymes include 3′-5 exonucleases, endonucleases (e.g. AP endonucleases), 3′-phosphodiesterase, and phosphatases, and are discussed below.

A preferred 3′ deblocking enzyme is a 3′-5′ exonuclease, such as preferably exonuclease III. Exonuclease III converts 3′-blocked open circular plasmid to 3′-hydroxyl gapped plasmid. This is accomplished either by the 3′-5′ exonuclease activity of exonuclease III, or by the other known 3′ deblocking activities of exonuclease III (3′-phosphatase, apurinic/apyrimidinic (AP) endonuclease and 3′-phosphodiesterase). When coincubated with DNA polymerase, the ratio of exonuclease III and DNA polymerase activities should be balanced appropriately to avoid significant exonuclease degradation of open circular plasmid. Exonuclease III has been characterized in numerous organisms. Other 3′-5 exonucleases may also serve as a 3′ deblocking enzyme, preferably having low processivity.

Exonuclease III at concentrations of about 0.5 units/I or higher may possibly offer an added benefit of selectively digesting some linear single stranded chromosomal DNA during the first reaction. The inventor suspects that exonuclease III is able to digest linear single-stranded DNA, although poorly. Since some of the residual linear chromosomal DNA in the plasmid solution is likely in single-stranded form, it is possible that exonuclease III may hydrolyze some of this linear single-stranded chromosomal DNA.

Another useful 3′ deblocking enzyme is an endonuclease, such as preferably AP endonuclease. AP endonuclease converts AP sites in open circular plasmid to 3′ hydroxyl gapped plasmid. AP endonucleases are found in many organisms. AP endonuclease from any source may be used. A preferred AP endonuclease is endonuclease IV. A preferred source of endonuclease IV is E. coli. Another useful AP endonuclease may be APE1 (Ranalli, J. Biol. Chem. 277:41715-41724, 2002; Izumi et al. Carcinogenesis 21:1329-1334, 2000). Other AP endonucleases or other types of endonucleases may also serve as 3′ deblocking enzymes.

Another useful 3′ deblocking enzyme is phosphatase, such as preferably 3′-phosphatase. 3′-Phosphatase efficiently dephosphorylates a 3′-phosphate blocking group to 3′-hydroxyl. 3′-Phosphatase may be useful with any DNA polymerase in the first reaction. 3′-Phosphatase may augment the repair activity of some DNA polymerases which have the ability to remove 3′-phosphate blocking groups. For example, the literature reports that DNA polymerase I (or Klenow) is able to remove a 3′-phosphate terminus, but this activity is poor (Zhang, Biochemistry 40:153-159, 2001). Another useful 3′-phosphatase 3′-deblocking enzyme is polynucleotide kinase-3′-phosphatase (PNKP). In addition to the 3′-phosphatase activity, the polynucleotide kinase activity of PNKP is able to convert 5′-hydroxyl termini to 5′-phosphate termini. Other phosphatases may also be useful.

Other 3′ deblocking enzymes can be used provided that they convert the blocked 3′ terminus of open circular plasmid to a 3′ hydroxyl terminus. The deblocking enzyme may be selected from one or more 3′-5′ exonucleases, endonucleases, 3′-phosphodiesterases, phosphatases, or other enzymes. More than one 3′ deblocking enzyme may be used during the first reaction. For example, the incubation may comprise a mixture of exonuclease m and PNKP. A 3′ deblocking enzyme may be especially advantageous when used with a DNA polymerase which lacks 3′-5 exonuclease activity. A 3′ deblocking enzyme may be used with a DNA polymerase which has 3′-5′ exonuclease activity, possibly enhancing repair efficiency. Example 2 demonstrates non-limiting embodiments using 3′ deblocking enzymes.

A 5′ deblocking enzyme may optionally be used to assist in the first reaction. The 5′ deblocking enzyme converts a blocked 5′-terminus to a 5′-phosphate terminus. The 5′ deblocking enzyme may be able to remove 5′ blocking groups which DNA polymerase is unable to remove. A preferred 5′ deblocking enzyme is flap endonuclease, an enzyme which is homologous to the 5′-3′ exonuclease of DNA polymerase I. In eukaryotes and archaeabacteria, DNA polymerase and flap endonuclease are employed for repair of some single strand breaks (Lieber, Bioessays 19:233-240, 1997; Kim, J. Biol. Chem. 273:8842-8848, 1998; Shu, Trends Biochem Sci. 23:171-173, 1998). Incubation with 5′ deblocking enzyme and DNA polymerase are preferably performed simultaneously, but could potentially also be performed sequentially in the order: 5′ deblocking enzyme followed by DNA polymerase. Non-limiting examples of 5′ deblocking enzymes of potential use may include 5′-3′ exonucleases, AP lyases, flap endonucleases or flap exonucleases (such as FEN1 or T5 exonuclease), and DNA deoxyribophosphodiesterases. These enzymes are well characterized in the art of DNA repair (Friedberg et al. DNA Repair and Mutagenesis, ASM Press, 1995). Other 5′ deblocking enzymes may potentially be used provided that they convert a blocked 5′ terminus of open circular plasmid to a 5′ phosphate terminus.

A 5′ deblocking enzyme may advantageously reduce unintentional strand displacement side reactions of DNA polymerase or remove the displaced strand. A 5′ deblocking enzyme may possibly also selectively digest some linear chromosomal DNA. More than one 5′ deblocking enzyme may be used in the first reaction. A 5′ deblocking enzyme may be especially advantageous when used with a DNA polymerase which lacks 5′ terminus repair activity, such as 5′-3′ exonuclease activity. A 5′ deblocking enzyme may be used with a DNA polymerase which has 5′ terminus repair activity, possibly enhancing repair efficiency. The first reaction may optionally employ both 5′ and 3′ deblocking enzymes, simultaneously or in any order, but preferably simultaneously with DNA polymerase incubation.

It will be appreciated that having repair capacity for both 3′ and 5′ termini of open circular plasmid is preferable to maximize the conversion efficiency, using either the inherent repair activity of the DNA polymerase (such as 3′-5′ exonuclease for the 3′ terminus; such as 5′-3′ exonuclease or lyase for the 5′ terminus) or a deblocking enzyme or both. This may be accomplished in the following manners:

(1) Preferably, the DNA polymerase has both 3′ and 5′ terminus repair activities (e.g. some DNA polymerase I enzymes). In this case, a 3′ deblocking or 5′ deblocking enzyme or both may optionally be added to possibly enhance repair efficiency.

(2) If the DNA polymerase lacks 3′ terminus repair activity and has 5′ terminus repair activity (e.g. Taq DNA polymerase, some eukaryotic DNA polymerases), then preferably the first reaction is performed using DNA polymerase and a 3′ deblocking enzyme. In this case, a 5′ deblocking enzyme may optionally be added to possibly enhance repair efficiency. For eukaryotic DNA polymerases, the 5′ deblocking enzyme flap endonuclease may be especially advantageous in assisting the inherent 5′ terminus lyase repair activity of some eukaryotic DNA polymerases, such as DNA polymerase beta (Wilson, Mut. Res; 407:203-215, 1998; Wilson, Mut. Res. 460:231-244, 2000). For example, the first reaction may be performed using AP endonuclease, DNA polymerase beta, and optionally flap endonuclease (Wilson, C.S.H. Symp. Quant. Biol., LXV:143-155, 2000).

(3) If the DNA polymerase has 3′ terminus repair activity and lacks 5′ terminus repair activity (e.g. some phage DNA polymerases), then preferably the first reaction is performed using DNA polymerase and 5′ deblocking enzyme. In this case, a 3′ deblocking enzyme may optionally be added to possibly enhance repair efficiency.

(4) If the DNA polymerase lacks both 3′ and 5′ terminus repair activities (e.g. mutant DNA polymerases), then preferably the first reaction is performed using DNA polymerase, 3′ deblocking enzyme, and 5′ deblocking enzyme.

For any given DNA polymerase, a person skilled in the art may optionally select appropriate 3′ deblocking and/or 5′ deblocking enzymes based on the known enzyme activities of the DNA polymerase, the known in vivo system of the DNA polymerase for single strand break repair, and the desired conversion efficiency of open circular to supercoiled plasmid. It will be appreciated that some DNA polymerases may be advantageous if the DNA polymerase functions in vivo in DNA repair. Preferably, at least one repair activity is provided in the first reaction for both the 3′ terminus and the 5′ terminus of open circular plasmid, using either the repair activity from the DNA polymerase or a deblocking enzyme or both.

Using the preferred mode of the first reaction, most or nearly all unligatable open circular plasmid can be converted to 3′-hydroxyl, 5′-phosphate nicked plasmid.

Alternate Mode: In an alternate conversion method, the unligatable open circular plasmid is incubated with polynucleotide kinase and 3′-phosphatase in the presence of nucleotide cofactor, preferably using the enzyme polynucleotide kinase-3′-phosphatase (PNKP). This will convert unligatable open circular plasmid which is 3′-phosphate, 5′-hydroxyl nicked plasmid to 3′-hydroxyl, 5′-phosphate nicked plasmid. The 3′-phosphatase converts a 3′-phosphate blocking group to 3′-hydroxyl. The polynucleotide kinase, in the presence of cofactor (usually ATP), converts the 5′-hydroxyl to 5′-phosphate. The result of the enzyme incubations is 3′-hydroxyl, 5′-phosphate nicked plasmid. The incubations with 3′-phosphatase and polynucleotide kinase are preferably performed simultaneously, but could also be performed sequentially in any order. Example 6 demonstrates a non-limiting embodiment of the alternate mode.

Using the alternate mode of the first reaction, at least some of the unligatable open circular plasmid can be converted to 3′-hydroxyl, 5′-phosphate nicked plasmid.

Other Modes: Two general methods for performing the first reaction are described above. It will be appreciated that any method for converting unligatable open circular plasmid to 3′-hydroxyl, 5′-phosphate nicked plasmid may be used. Other methods may be provided using the many enzymes and methods known in the art of DNA repair (Friedberg et al. DNA Repair and Mutagenesis, ASM Press, 1995).

Second Enzymatic Reaction: Conversion of 3′-hydroxyl, 5′-phosphate Nicked Plasmid to Relaxed Covalently Closed Circular Plasmid.

In the second enzymatic conversion reaction (second reaction), the 3′-hydroxyl, 5′-phosphate nicked plasmid is converted in vitro to relaxed covalently closed circular plasmid. This is accomplished by incubation with a DNA ligase in the presence of DNA ligase nucleotide cofactor.

Third Enzymatic Reaction: Conversion of Relaxed Covalently Closed Circular Plasmid to Negatively Supercoiled Plasmid.

In the third enzymatic conversion reaction (third reaction), the relaxed covalently closed circular plasmid is converted in vitro to negatively supercoiled plasmid. This is accomplished by incubation with a DNA gyrase in the presence of DNA gyrase nucleotide cofactor (usually ATP).

The repair of open circular plasmid in a plasmid preparation has not been previously demonstrated experimentally. The nature of the DNA damage in open circular plasmid in plasmid preparations has not been investigated in the literature. To date, no one has experimentally demonstrated that this open circular plasmid can be converted to supercoiled plasmid in vitro. This is the first demonstration that such open circular plasmid can be converted in vitro. Surprisingly and unexpectedly, in the preferred mode, the conversion of open circular plasmid to supercoiled plasmid may be nearly quantitative. Nearly all of the open circular plasmid may be converted to supercoiled plasmid.

### Performing Enzymatic Conversion Reactions

The three enzymatic conversion reactions are preferably performed simultaneously in a single combined incubation, using an enzyme mixture. In a preferred mode, the enzyme mixture may comprise DNA polymerase, DNA ligase, and DNA gyrase. This mixture may further comprise one or more 3′ deblocking enzymes, such as exonuclease III, AP endonuclease, 3′-phosphatase, or PNKP. This mixture may further comprise one or more 5′ deblocking enzymes. In an alternate mode, the enzyme mixture may comprise 3′-phosphatase, polynucleotide kinase, DNA ligase, and DNA gyrase. By using a single combined incubation, open circular plasmid unintentionally generated during an incubation (e.g. by an enzyme contaminant) may be converted to supercoiled plasmid. Alternatively, the three enzymatic conversion reactions may also be performed sequentially in the order: first reaction, second reaction, and third reaction. Alternatively, the first and second reactions may be performed simultaneously, followed by the third reaction. Alternatively, the first reaction may be performed, followed by the second and third reactions simultaneously. Such sequential reaction may or may not be separated by isolating total plasmid from the rest of the reaction mixture.

The enzymatic conversion reactions may be performed with intermediate purification of plasmid. For example, after the second reaction, plasmid could be purified by chromatography. The purified plasmid could subsequently be incubated with DNA gyrase in the third reaction. A disadvantage of such intermediate purification embodiments is that a substantial amount of plasmid may be lost in the intermediate purification. Preferably, the enzymatic conversion reactions are performed without intermediate purification of plasmid. For example, the second reaction is preferably performed without prior purification of plasmid after the first reaction. The third reaction is preferably performed without prior purification of plasmid after the second reaction.

If the optimal incubation conditions, such as temperature or pH or buffer conditions, differ for the enzymes used herein, it may be advantageous to perform the enzymatic conversion reactions sequentially. For example, assume that DNA polymerase and DNA ligase have an optimal incubation temperature of 37° C., and DNA gyrase is derived from a thermophile with an optimal incubation temperature of 55° C. In this embodiment, the first and second reactions are preferably performed at 37° C. The temperature is then preferably increased to 55° C. for the DNA gyrase incubation (i.e., third reaction).

For some applications, relaxed covalently closed circular plasmid may have the same bioactivity as supercoiled plasmid. In this case, the third reaction with DNA gyrase may be omitted. If the second reaction with DNA ligase is performed in the presence of an intercalating agent, then removal of the intercalating agent after ligation will result in negatively supercoiled plasmid. Preferably, the second reaction is performed in the absence of an intercalating agent, due to the carcinogenic nature of intercalating agents.

Preferably, the enzymatic conversion reactions will convert at least 60%, at least 70%, at least 80%, at least 90%, or at least 95% of open circular plasmid in the plasmid solution to supercoiled plasmid.

### Enzymes

3′-Phosphatase and polynucleotide kinase enzymes from any source may be used provided that they are active on open circular plasmid substrate. Polynucleotide kinase and 3′-phosphatase enzyme activities are sometimes found on a single polypeptide in some organisms, known as polynucleotide kinase-3′-phosphatase (PNKP). PNKP helps repair single strand breaks in double stranded DNA. PNKP has been characterized in numerous organisms, including rats, human, bovine, plasmodium, S. pombe, and mouse (Karimi-Busheri et al., Nucl. Acids Res. 26:4395-4400, 1998). 3′-Phosphatase with no associated polynucleotide kinase activity has been characterized in the yeast Saccharomyces cereviseae and the plant Arabidopsis thaliana (Vance et al., J. Biol. Chem. 276:15073-15081, 2001). Polynucleotide kinase with no associated 3′-phosphatase could potentially be obtained by mutation of PNKP. The polynucleotide kinase and 3′-phosphatase enzymes can be present on the same protein (PNKP) or on separate proteins. Preferably, the two enzymatic activities are present on the same protein. A preferred source of PNKP is human.

DNA polymerases from any source may be useful: e.g. Klenow DNA polymerase, eubacterial DNA polymerases, phage DNA polymerases, viral DNA polymerases, eukaryotic DNA polymerases, archaebacterial DNA polymerases, and genetically mutated versions thereof. A preferred DNA polymerase has both 3′-5′ and 5′-3′ exonuclease activities, such as DNA polymerase I from some sources. Preferably, the DNA polymerase is substantially not strand displacing on a nicked plasmid template, but instead hydrolyzes the strand by its 5′-3′ exonuclease activity. The inventor has observed that DNA polymerase I, in the presence of deoxyribonucleoside triphosphate substrates, converts most of the open circular plasmid to 3′-hydroxyl, 5′-phosphate nicked plasmid. DNA polymerases lacking inherent 5′-3′ exonuclease activity in combination with a 5′-3′ exonuclease may function in an equivalent manner to DNA polymerase I. DNA polymerase I, or its equivalents, is likely found in many organisms. A preferred source of DNA polymerase I is E. coli.

Exonuclease III from any source may be useful. Exonuclease m, or other closely related 3′ deblocking enzymes, is likely found in many organisms. Exonuclease III is especially useful in combination with DNA polymerase I to deblock the 3′ terminus of 3′ blocked open circular plasmid, enhancing the conversion efficiency. Exonuclease m has four activities, all of which may serve a deblocking function: 3′-5′ exonuclease activity, 3′-phosphatase activity, apurinic/apyrimidinic (AP) endonuclease activity and 3′-phosphodiesterase. A preferred source of exonuclease III is E. coli.

DNA ligase from any source may be used, provided that it is capable of ligating 3′-hydroxyl, 5′-phosphate nicks. DNA ligase is found in many organisms. DNA ligases from bacteriophages, viruses, eukaryotes, and archaebacteria usually require adenosine triphosphate (ATP) as the nucleotide cofactor. DNA ligases from eubacteria, such as E. coli, usually require nicotinamide adenine dinucleotide (NAD) as the cofactor. It will be appreciated that equivalent cofactors could be used. For example, dATP could be used in place of ATP for some ligases. Preferably, the DNA ligase requires ATP cofactor. A preferred source of DNA ligase is bacteriophage T4.

DNA gyrase from any source can be used, provided that it converts relaxed covalently closed circular plasmid to supercoiled plasmid. DNA gyrase is found in eubacteria and some archeabacteria. DNA gyrase converts relaxed covalently closed circular plasmid to negatively supercoiled plasmid in the presence of ATP or an equivalent nucleotide. A preferred source of DNA gyrase is E. coli. Another useful source of DNA gyrase could be Vibrio cholera. Vibrio cholera DNA gyrase is reported to be unable to catalyze the reverse reaction (Mukhopadhyay et al., Biochemical J. 280:797-800, 1991).

The incubation with DNA gyrase is preferably performed substantially in the absence of topoisomerase I, which converts supercoiled plasmid to relaxed covalently closed circular plasmid. The presence of topoisomerase I during the DNA gyrase incubation could reduce the extent of supercoiling by DNA gyrase. It will be appreciated that enzyme purity is rarely absolute. Topoisomerase I may be considered absent, in a functional sense, if it is present at such a low level that it does not significantly affect the extent of supercoiling by DNA gyrase. The DNA gyrase incubation could be performed in the presence of an inhibitor specific for topoisomerase I, reducing the detrimental effect of any topoisomerase I contamination.

Reverse DNA gyrase may be used instead of DNA gyrase. Reverse DNA gyrase is found in many thermophilic bacteria. Reverse DNA gyrase converts relaxed covalently closed circular plasmid to positively supercoiled plasmid. The use of reverse DNA gyrase would produce a plasmid preparation of positively supercoiled plasmid. Preferably, however, DNA gyrase is employed, because negatively supercoiled plasmid is known to be biologically active in human cells.

### Repair Enzymes and Accessory Proteins

The repair of single strand breaks in double stranded DNA is an essential function of the DNA repair system of all living organisms. Numerous repair enzymes and accessory proteins are known which facilitate the repair of single strand breaks of all types. Such enzymes and accessory proteins could be used to accelerate or improve the conversion of unligatable open circular plasmid to covalently closed circular plasmid. Non-limiting examples of other proteins/enzymes of potential use in repairing single stranded breaks in open circular plasmid may include protein HU, XRCC1, RNase H, DNA glycosylases, damage-specific endonucleases (e.g. UvrABC), and enzymes involved in single strand break repair, base excision repair, nucleotide excision repair, or mismatch repair (Friedberg et al. DNA Repair and Mutagenesis, ASM Press, 1995).

### Optional Nucleotide Cofactor Regeneration

Several enzymes used herein require nucleotide cofactors. DNA gyrase requires ATP for activity, generating ADP as the nucleotide cofactor by-product. Polynucleotide kinase requires ATP for activity, generating ADP as the nucleotide cofactor by-product. DNA ligase requires ATP (or NAD) for activity, generating AMP (or NMP) as the nucleotide cofactor by-product. Optionally, the nucleotide cofactor by-product may be enzymatically converted back to nucleotide cofactor during one or more of the reactions, thus, helping to maintain a constant concentration of ATP (or NAD).

In the third reaction, incubation with DNA gyrase generates ADP as the nucleotide cofactor by-product. Optionally, ADP can be converted back to ATP during the DNA gyrase incubation using a kinase enzyme and a high energy phosphate donor (i.e., the kinase enzyme substrate). The preferred kinase and phosphate donor are pyruvate kinase and phosphoenolpyruvate (PEP). The pyruvate kinase and PEP are coincubated with DNA gyrase to help maintain a constant ATP concentration. Fructose diphosphate may be added as an allosteric activator of pyruvate kinase. Another kinase and high energy phosphate donor may be creatine kinase and creatine phosphate. Yet another kinase and high energy phosphate donor may be acetate kinase and phosphoacetate. This method can optionally also be employed in the polynucleotide kinase incubation in the alternate mode of the first reaction to convert ADP, the nucleotide cofactor by-product, back to ATP.

In the second reaction, incubation with DNA ligase generates AMP as the nucleotide cofactor by-product. Optionally, AMP can be converted back to ATP during the DNA ligase incubation using a mixture of adenylate kinase, pyruvate kinase, and PEP. Adenylate kinase converts AMP to ADP in the presence of ATP. Pyruvate kinase and PEP convert ADP to ATP. Adenylate kinase, pyruvate kinase, and PEP are coincubated with DNA ligase to help maintain a constant ATP concentration. If the cofactor for DNA ligase is NAD, the nucleotide cofactor by-product nicotinamide monophosphate (NMP) can be converted back to NAD by the enzyme nicotinamide adenylyltransferase. AMP generated by the latter enzyme could be converted back to ATP as described.

Pyrophosphate is generated as a by-product of the DNA ligase and the DNA polymerase reactions. Optionally, inorganic pyrophosphatase may be included during the incubation with DNA ligase and/or DNA polymerase, to hydrolyze pyrophosphate to phosphate.

DNA polymerase may generate dNMP by-products. The dNMP by-products could optionally be enzymatically converted back to dNTPs. This is accomplished using the enzymes cytidylate kinase, thymidylate kinase, adenylate kinase, guanidylate kinase, and nucleoside diphosphate kinase. For example, dCMP is converted to dCDP by cytidylate kinase, which is then converted to dCTP by nucleoside diphosphate kinase (NDK).

In an alternate mode, the enzymatic conversion reactions may be performed in a single incubation using a mixture of 3′-phosphatase, polynucleotide kinase, DNA ligase, and DNA gyrase. If the latter three enzymes require ATP cofactor, then adding adenylate kinase, pyruvate kinase, and PEP to this incubation could help maintain a constant ATP concentration.

The use of enzymes for regenerating nucleotide cofactor from their nucleotide by-product is optional. To the inventor's knowledge, the use of nucleotide cofactor regeneration to improve the enzymatic activity of DNA ligase or polynucleotide kinase is not known in the prior art. Example 3 demonstrates a non-limiting embodiment using ATP regeneration.

### Optional Reaction with Exonuclease

An optional additional in vitro enzymatic reaction with exonuclease may be performed to selectively hydrolyze residual linear chromosomal DNA contamination in the plasmid solution, preferably without substantially hydrolyzing covalently closed circular plasmid. Additionally, in some embodiments, the exonuclease reaction may advantageously degrade open circular plasmid which is remaining after the second reaction. The result of the exonuclease reaction is a reduction in the chromosomal DNA contamination in the final plasmid preparation. Complete exonuclease digestion of linear chromosomal DNA to undetectable levels may not be necessary. Instead, the exonuclease reaction may reduce chromosomal DNA contamination to a lower level. It will be appreciated that the selectivity of the exonuclease need not be absolute, and most exonucleases lack absolute substrate specificity. A small loss of plasmid due to lack of absolute substrate specificity by an exonuclease may be acceptable or perhaps necessary to achieve desired reduction in chromosomal DNA. One or more exonucleases may be used for the exonuclease reaction. Alternatively, the linear chromosomal DNA may be reacted with one or more exonucleases, wherein said exonucleases have at least some substrate selectivity in preferentially degrading linear chromosomal DNA substrate versus covalently closed circular plasmid substrate, whereby at least some linear chromosomal DNA is degraded. In some cases, a substantial loss of plasmid may possibly result due to lack of absolute substrate specificity. Exonucleases are known (e.g. Isfort, BioTechniques 12:800-803, 1992) but have not previously been used in this context of converting open circular plasmid to supercoiled plasmid.

The selection of the exonuclease(s) depends on when the reaction is performed. If the reaction with exonuclease(s) is performed prior to completing the second reaction, the exonuclease(s) preferably should selectively degrade the linear chromosomal DNA, substantially without degrading open circular plasmid, relaxed covalently closed circular plasmid, and supercoiled plasmid. Non-limiting examples of such exonucleases may include exonuclease I, lambda exonuclease, exonuclease V, exonuclease VII, exonuclease VIII, exonuclease T (RNase T), recJf, or combinations thereof. Such exonucleases may be conveniently used concurrently with all the enzymatic conversion reactions. In addition, deblocking enzymes which are also exonucleases may potentially serve a dual function of hydrolyzing chromosomal DNA. It will be appreciated that a small amount of plasmid (such as open circular plasmid or closed circular plasmid) may potentially be degraded by the exonuclease reaction, due to a lack of absolute enzyme substrate specificity. Alternatively, if the exonuclease reaction is performed prior to completing the second reaction, the linear chromosomal DNA may be reacted with one or more exonucleases, wherein said exonucleases have at least some substrate selectivity in preferentially degrading linear chromosomal DNA substrate versus open circular and covalently closed circular plasmid substrates, whereby at least some linear chromosomal DNA is degraded. In some cases, a substantial loss of plasmid may possibly result due to lack of absolute substrate specificity. The optional exonuclease reaction is preferably performed concurrently with the enzymatic conversion reactions, preferably using exonuclease V. Example 4 demonstrates non-limiting embodiments using concurrent exonuclease digestion.

If the reaction with exonuclease(s) is performed after the second reaction, the exonuclease(s) preferably should selectively degrade linear chromosomal DNA, substantially without degrading relaxed covalently closed circular plasmid and supercoiled plasmid. Non-limiting examples of such exonucleases may include exonuclease I, exonuclease III, exonuclease V, exonuclease VII, exonuclease VIII, lambda exonuclease, T7 exonuclease, T5 exonuclease, exonuclease T, RecJf, or combinations thereof. DNA polymerase I may be used as an exonuclease in the absence of deoxyribonucleoside triphosphate substrates. An especially advantageous combination comprises exonuclease I and exonuclease III. Such exonucleases may be conveniently used subsequent to the enzymatic conversion reactions. A small amount of covalently closed circular plasmid may potentially be degraded by the exonuclease reaction due to a lack of absolute exonuclease substrate specificity. It will be appreciated that the conversion of open circular plasmid to covalently closed circular plasmid in the enzymatic conversion reactions will usually not be 100%, resulting in remaining open circular plasmid after the second reaction. In one embodiment, after the second reaction, this exonuclease reaction may also advantageously degrade this remaining open circular plasmid using an exonuclease which degrades open circular plasmid, for example using exonuclease III. If DNA polymerase and deoxyribonucleoside triphosphate substrates are present during this exonuclease digestion using exonuclease III, then the concentration of exonuclease III should be adjusted appropriately to effect digestion of linear double stranded chromosomal DNA. Alternatively, DNA polymerase (and/or other enzymes used in the enzymatic conversion reactions) may optionally be inactivated prior to the subsequent exonuclease digestion, such as by heat inactivation. Alternatively, if the exonuclease reaction is performed after the second reaction, the linear chromosomal DNA may be reacted with one or more exonucleases, wherein said exonucleases have at least some substrate selectivity in preferentially degrading linear chromosomal DNA substrate versus covalently closed circular plasmid substrate, whereby at least some linear chromosomal DNA is degraded. In some cases, a substantial loss of covalently closed circular plasmid may possibly result due to lack of absolute substrate specificity. Example 5 demonstrates a non-limiting embodiment using subsequent exonuclease digestion.

The amount of plasmid degraded during the exonuclease reaction may depend on factors such as substrate specificity of the exonuclease(s) employed, whether the exonucleases are used to remove remaining open circular plasmid after the second reaction and the amount of such remaining open circular plasmid, and reaction conditions (enzyme concentrations, incubation times, etc). The plasmid loss is preferably not substantial; however, in some cases, the loss may be substantial. For example, plasmid loss may be substantial if a large amount of open circular plasmid is remaining after the second reaction, and this remaining open circular plasmid is degraded by exonuclease. Plasmid loss may be minimized by using high specificity exonucleases or high efficiency enzymatic conversion reactions or both.

In one embodiment, the exonuclease reaction may be performed using one or more single stranded DNA exonucleases, such as exonuclease I. Thus, if some chromosomal DNA is in single stranded form, then such exonuclease reaction may reduce chromosomal DNA contamination. Some single stranded chromosomal DNA may have a 3′ blocking group, such as 3′-phosphate, which prevents exonuclease digestion. The 3′ deblocking enzyme used in the first reaction (such as exonuclease III or 3′-phosphatase) may potentially act synergistically, by converting 3′-blocked single stranded chromosomal DNA to a 3′ hydroxyl terminus, which is suitable for exonuclease digestion. Experiments by the inventor suggest that some linear chromosomal DNA contamination from an alkaline lysis cleared lysate can be digested by exonuclease I. Optionally, double stranded linear chromosomal DNA could be converted to single stranded form by a brief denaturation step after the second reaction and prior to exonuclease digestion.

In another embodiment, the exonuclease reaction may be performed using a combination of one or more single stranded DNA exonucleases and one or more double stranded DNA exonucleases. One advantageous combination comprises exonuclease I and exonuclease III.

In another embodiment, one or more exonucleases is incubated concurrently with the enzymatic conversion reactions (such as exonuclease I). After the second reaction, one or more additional exonucleases is then added to further digest chromosomal DNA (such as exonuclease III).

Additional enzymes, such as polynucleotide kinase and/or 3′-phosphatase, may be useful in converting the termini of linear chromosomal DNA to the desired phosphorylation state to facilitate exonuclease digestion. Optionally, after a conversion reaction, plasmid may be purified prior to the exonuclease reaction. Preferably though, after a conversion reaction, plasmid is not purified prior to the exonuclease reaction.

A preferred exonuclease is exonuclease V, also known as ATP dependent exonuclease, preferably an exonuclease V with low helicase activity such as M. luteus. Exonuclease V hydrolyzes linear chromosomal DNA to small oligonucleotides or nucleotides. This enzyme requires the cofactor ATP, generating ADP as the nucleotide cofactor by-product. The use of exonuclease V is synergistic. The reaction with exonuclease V could be performed in the presence of a kinase enzyme and high energy phosphate donor which converts ADP nucleotide cofactor by-product back to ATP as described above. In one embodiment, the enzymatic conversion reactions may be performed in a single incubation using a mixture of enzymes: DNA polymerase, DNA ligase, DNA gyrase, exonuclease V, and optionally regenerating enzymes which convert AMP or ADP or both (the nucleotide cofactor by-products) back to ATP (such as adenylate kinase, pyruvate kinase, and PEP). This enzyme mixture may optionally further comprise a 3′ deblocking enzyme, such as exonuclease III. To the inventor's knowledge, the use of ATP regeneration during digestion by exonuclease V is not known in the prior art. A preferred source of exonuclease V is M. luteus.

It is conceivable, but highly unlikely, that the oligonucleotide products of exonuclease V hydrolysis could be polymerized by DNA ligase. The oligonucleotide products are likely poor substrates for DNA ligase. If polymerization does occur to a significant extent, the problem could be addressed by: (a) increasing the concentration of exonuclease V, (b) using a DNA ligase which is unable to ligate blunt ends, (e.g. E. coli DNA ligase), (c) adding an additional exonuclease (e.g. such as exonuclease I) to hydrolyze the oligonucleotides to nucleotides, (d) performing the exonuclease digestion after the incubation with DNA ligase, or (e) combinations thereof.

The use of exonucleases for selective hydrolysis of chromosomal DNA in combination with conversion of open circular plasmid to supercoiled plasmid works synergistically to overcome the limitations of prior art uses of exonucleases. Prior exonuclease digestion methods for removing chromosomal DNA fall into two categories. In one prior approach, exonucleases hydrolyze both chromosomal DNA and open circular plasmid. The disadvantage of this approach is that open circular plasmid is degraded. In the other prior approach, exonucleases hydrolyze only chromosomal DNA, leaving supercoiled and open circular plasmid intact. The disadvantage of this approach is that open circular plasmid must be removed by subsequent purification. The combination of exonuclease digestion of chromosomal DNA and conversion of open circular plasmid to supercoiled plasmid overcomes these disadvantages of the prior art. A single incubation could potentially produce high purity supercoiled plasmid with low levels of contaminating chromosomal DNA without significant loss of plasmid. The optional exonuclease reaction may be especially advantageous for low copy plasmids, which tend to have a higher percentage of chromosomal DNA contamination than high copy plasmids. The optional exonuclease reaction may be useful in combination with any method which converts open circular plasmid to supercoiled plasmid.

Optionally, a ribonuclease could be used to hydrolyze residual RNA. Incubation with ribonuclease could be performed at any time. The reaction with ribonuclease could be performed as a separate incubation or simultaneously with another enzymatic reaction. Ribonuclease is known but has not previously been used in this context of converting open circular plasmid to supercoiled plasmid. A preferred ribonuclease is ribonuclease I.

Undesired plasmid optionally may be removed by selective restriction endonuclease digestion. If two or more plasmids are present in a plasmid solution, usually only one plasmid is the desired product. For example, a host cell may contain two different plasmids. Alternatively, two different plasmids could be generated from one plasmid by incubation with a recombinase. The resulting selectively linearized undesired plasmid could be further hydrolyzed by incubation with an exonuclease(s). It will be appreciated that the use of restriction enzyme in this manner does not involve degradation of the plasmid of interest.

Temperature may advantageously be used as an on/off switch of enzyme activity. For example, the enzymatic conversion reactions may be performed at 37° C. using E. coli enzymes. After converting open circular plasmid to supercoiled plasmid, the temperature could be increased to 60° C. for selective degradation of chromosomal DNA using thermophilic exonuclease(s), such as exonuclease I and/or III. At 60° C., the E. coli enzymes are likely to be inactive. At 37° C., the thermophilic exonuclease(s) may be poorly active, and thus not interfere with the reactions of the E. coli enzymes.

### Optional Reaction with a Potent Decatenase

DNA gyrase is known to reversibly catalyze the formation of catenanes (Kreutzer, Cell 20:245-254, 1980; Krasnow, J. Biol. Chem. 257:2687-2693, 1982). A catenane is formed by interlocking of two or more plasmid molecules, forming dimers or multimers. According to the prior art, the supercoiled plasmid may form dimers, trimers, and higher multimers; open circular plasmid and relaxed covalently closed circular plasmid may form huge multimeric catenanes, comprising thousands of interlocked plasmid molecules. The formation of catenanes may be potentially counter-productive for gene transfer. Due to the much larger molecular size of dimers and other multimers, catenanes could possibly have reduced transformability and bioactivity. Significant catenane formation could potentially produce a plasmid preparation with less bioactivity than the preparation prior to the enzymatic conversion reactions. Preferably, the incubation with DNA gyrase is performed to avoid or to minimize formation of catenanes. This may be accomplished by appropriate selection of the cation buffer composition, such as the spermidine concentration, as taught in prior art. The spermidine concentration may be lowered to avoid or minimize catenane formation. Potentially, the buffer composition could be selected such that the amount of catenanes in the plasmid solution would be reduced by the DNA gyrase incubation. Conversely, for applications in which catenanes are desirable, the buffer composition could be potentially selected such that the amount of catenanes would be increased by the DNA gyrase incubation.

In the examples, no significant catenation was observed. Even at the highest plasmid concentration in Example 1 of 1.5 μg/l, no significant catenation was observed. Based on visual inspection of agarose gels in the examples, it is estimated that the amount of catenane formation is less than approximately 1% to 5% of total plasmid. If catenation does occur, the amount of catenane formed is probably insignificant for most applications. Preferably, the enzymatic conversion reactions are performed to avoid or minimize the formation of catenanes. Preferably, the amount of catenane formation resulting from the third reaction is less than 1%, less than 5%, less than 10%, less than 15%, or less than 20% of the total plasmid; preferably, without the use of a potent decatenase.

If catenane formation does occur to an undesirable extent, then catenane formation could be reduced by several possible methods. In a first method, the DNA gyrase-containing reaction could be performed at a lower plasmid concentration or performed in a manner that minimizes plasmid aggregation. In a second method, a DNA gyrase with stronger decatenase activity could be used, such as Mycobacterum smegmatis DNA gyrase. In a third method, catenation could be reduced or eliminated by an optional additional incubation with a potent decatenase enzyme. The incubation with a potent decatenase is preferably performed simultaneously with the DNA gyrase reaction, but could be performed after the DNA gyrase reaction. Topoisomerase III and topoisomerase IV are potent decatenases; both potent decatenases relax supercoiled plasmid at a slow rate. Therefore, one or more potent decatenases could be used at a minimal concentration, to effect decatenation and to minimize supercoiled relaxation. A preferred potent decatenase is topoisomerase IV. Both potent decatenases convert ATP nucleotide cofactor to ADP. This optional incubation could be performed in the presence of a kinase enzyme and high energy phosphate donor to convert ADP back to ATP. Preferably though, incubation with a potent decatenase is not performed.

### Plasmid Recovery

After the enzymatic conversion reactions, the resulting plasmid may be used directly in some applications without further purification. For other applications, after the enzymatic conversion reactions, additional plasmid purification may be desirable, for example to remove the buffer salts, enzymes, nucleotides, or possibly exonuclease hydrolysis products. This can be accomplished by many methods such as, for example, organic solvent extraction, chromatography (e.g. gel filtration, anion exchange, hydrophobic interaction, reverse phase), precipitation, ultrafiltration, ultracentrifugation, electrophoresis, or combinations thereof. The additional purification may also remove residual open circular plasmid. This can be accomplished using methods such as chromatography, electrophoresis, ultracentrifugation, or exonuclease hydrolysis. The additional purification may also remove residual linear chromosomal DNA.

The recovered supercoiled plasmid will likely be a mixture of supercoiled plasmid produced using the enzymatic conversion reactions and supercoiled plasmid originally present in the cleared lysate.

In one advantageous embodiment, plasmid from a cleared lysate is purified chromatographically prior to the enzymatic conversion reactions. After such enzymatic conversion reactions, the plasmid product is purified chromatographically (preferably using the same chromatographic column) as a final “polishing” procedure. The chromatographic method is preferably anion exchange, before and after the conversion reactions, such as an anion exchange column for plasmid purification (Qiagen, Macherey-Nagel).

Applications for the recovered supercoiled plasmid may include transformation into recipient competent cells, such as tissue culture or whole animals, and especially for human therapeutic use (i.e., treatment of existing disease or prevention of disease). When the conversion reactions are used in combination with the optional exonuclease reaction, the final plasmid product may have a high percentage of supercoiled plasmid and a low percentage of chromosomal DNA contamination.

### Optional Reuse of Enzyme

In one embodiment, one or more of the enzymes could be covalently attached to a solid support. The resulting enzyme-solid support could be packed in a chromatography column, producing an enzyme column. An immobilized enzyme column could be made for each enzyme in the method separately; alternatively, a single immobilized enzyme column could contain a mixture of enzymes to convert unligatable open circular plasmid to supercoiled plasmid. Plasmid solution could be run through the column or series of columns, converting unligatable open circular plasmid to supercoiled plasmid. Column eluate could be recycled through the column(s) as needed until most of the unligatable open circular plasmid is converted to supercoiled plasmid. An immobilized enzyme column could be used multiple times to prepare multiple plasmids with appropriate washing before reuse. Preferably, however, the enzymes are not attached to a solid support and are free in solution.

For bulk scale plasmid preparations, large quantities of enzymes may be needed. Producing a large quantity of enzyme may be expensive. In this situation, it may be optionally advantageous to recover the enzymes after the incubation so that the enzymes could be reused for subsequent plasmid preparations. To recover the enzyme(s) for reuse, the enzyme(s) must be separated from the plasmid. This could be performed by using affinity chromatography if the enzymes have an affinity tag, such as polyhistidine. This could also be performed using classical chromatography, such as anion or cation exchange, which would separate the plasmid from the enzymes. If the enzymes are recovered after the incubation, loss of enzyme activity should be minimized during incubation. This could be accomplished by lowering the incubation temperature slightly or by adding stabilizers of enzyme activity, such as glycerol, Triton X-100, spermidine, bovine serum albumin, or dithiothreitol.

In one advantageous embodiment, one or more enzymes used may be thermostable and derived from a thermophilic organism. Recombinant thermostable enzymes are readily purified from E. coli, since E. coli proteins are unstable at higher temperatures. For example, some or all of the enzymes could be derived from a thermophilic prokaryote, such as Bacillus stearothermophilus or Thermotoga maritima, or a thermophilic eukaryote, such as Thermomyces lanuginosus. The incubations with thermostable enzyme could be performed at temperatures between about 50° C. and 75° C. Thermostable enzymes would likely maintain most of their activity during the incubation, optionally allowing reuse for subsequent incubations if desired.

### Reactions Preferably not Performed

It will be appreciated that one objective is to produce a plasmid preparation with a higher percentage of supercoiled plasmid than its initial percentage prior to the enzymatic conversion reactions. Therefore, additional reactions which work against this objective are preferably not performed. Most plasmid preparations contain a mixture of supercoiled and open circular plasmid prior to the enzymatic conversion reactions. Therefore, it is advantageous to preserve the supercoiled plasmid in the plasmid solution prior to and during the incubation(s). After preparing a cleared lysate, the enzymatic conversion reactions are preferably, in advantageous embodiments, performed without prior purposeful in vitro conversion of supercoiled plasmid to an undesired form, or without such purposeful in vitro conversion during the enzymatic conversion reactions. Undesired forms include linear, open circular, relaxed covalently closed circular, replicated daughter plasmids (partial or complete), single stranded circular, triple stranded, single-strand invasion, or Holliday structure forms. After preparing the cleared lysate, the first and second (and optionally the third in some embodiments) reactions are preferably, in advantageous embodiments, performed without prior purposeful separation of supercoiled plasmid from the open circular plasmid.

It will be appreciated that one objective is to include derivatives of open circular plasmid in the final plasmid preparation by converting open circular plasmid to supercoiled plasmid. In this manner, preferably, the enzymatic conversion reactions increase the amount of supercoiled plasmid without increasing the total amount of plasmid. It is preferable to preserve the open circular plasmid prior to the enzymatic conversion reactions so that it may be quantitatively converted to supercoiled plasmid. After preparing a cleared lysate, the first and second (and optionally the third in some embodiments) reactions are preferably, in advantageous embodiments, performed without purposeful in vitro conversion of open circular plasmid to an undesired form, or without prior purposeful in vitro conversion of open circular plasmid to an undesired form. Undesired forms include forms other than covalently closed circular plasmid or forms with impaired ability to be subsequently converted to covalently closed circular plasmid. Such undesired forms include linear, single stranded circular, triple stranded, single-strand invasion, in vitro replicated daughter plasmids (partial or complete), or Holliday structure forms. Preferably, after preparing a cleared lysate, and prior to the enzymatic conversion reactions, open circular plasmid is not purposefully separated from supercoiled plasmid.

It will be appreciated that some plasmid may be degraded in the optional exonuclease reaction. Such degradation is not considered purposeful, since the purpose of the exonuclease reaction is degradation of chromosomal DNA and/or degradation of remaining open circular plasmid after the second reaction.

The following embodiments may be especially advantageous. After preparing a cleared lysate, the cleared lysate usually comprises supercoiled plasmid in addition to open circular plasmid. After preparing a cleared lysate, the supercoiled plasmid is preferably not purposefully modified prior to the enzymatic conversion reactions. Purposeful modification is usually a quantitative conversion, in which most of the material is converted to a different form. Preferably, after preparing a cleared lysate and prior to the enzymatic conversion reactions, supercoiled plasmid from the cleared lysate is not purposefully converted to open circular plasmid, for example by intentional free radical nicking, incubation with a nickase such as NBstBI, or DNase I nicking. Preferably, after preparing a cleared lysate and prior to the enzymatic conversion reactions, supercoiled plasmid from the cleared lysate is not purposefully converted to relaxed covalently closed circular plasmid, for example by incubation with topoisomerase I or with DNA ligase and AMP. Preferably, after preparing a cleared lysate and prior to the enzymatic conversion reactions, supercoiled plasmid (or open circular plasmid) is not purposefully converted to linear form, for example by restriction digestion. Preferably, after preparing the cleared lysate and prior to the enzymatic conversion reactions, open circular plasmid in the plasmid solution is not purposefully converted to single stranded circular DNA, for example by heating.

Preferably, after preparing a cleared lysate and prior to the enzymatic conversion reactions, the nucleotide sequence of the plasmid is not modified.

Preferably, after preparing a cleared lysate, the enzymatic conversion reactions are performed without purposeful in vitro plasmid replication and without prior purposeful in vitro plasmid replication. “In vitro plasmid replication” is defined herein as enzymatic production of daughter plasmid molecules (either partial or complete synthesis) from a parent plasmid in vitro. Partial production of daughter molecules on some plasmids begins with initiation of new strand synthesis and produces a theta structure as viewed with an electron microscope. Partial production of daughter molecules by rolling circle replication results in production of single stranded molecules from the parent plasmid. It will be appreciated that in the first reaction, DNA polymerase may generate a small amount of displaced single stranded DNA by strand displacement as an unintentional side reaction of DNA repair of open circular plasmid, not as intentional plasmid replication. Such flaps may potentially be repaired using a flap endonuclease. An example of in vitro plasmid replication is described by Funnel et al. (J. Biol. Chem. 261:5616-5624, 1986). Preferably, in vitro plasmid replication is not performed after the enzymatic conversion reactions.

Preferably, the enzymatic conversion reactions are performed without an in vitro incubation, or prior in vitro incubation, with a primase enzyme or an RNA polymerase enzyme, which produces primers for synthesis of daughter strands of plasmid.

Preferably, the plasmid solution does not comprise unligatable open circular plasmid which was synthesized by a purposeful in vitro method, such as in vitro enzymatic or nonenzymatic DNA synthetic reactions. Non-limiting examples of in vitro synthesis include in vitro replication forming open circular daughter plasmids, open circular plasmid synthesized from single stranded circular DNA by in vitro enzymatic reactions or synthetic primer annealing, and purposeful damage of supercoiled plasmid with free radicals to produce unligatable open circular plasmid. Preferably, the enzymatic conversion reactions are performed without purposeful in vitro synthesis of open circular plasmid, for example from nucleic acid which is not open circular plasmid. Preferably, the plasmid solution does not comprise open circular plasmid which was purposefully synthesized in vitro.

Preferably, the enzymatic conversion reactions are performed without increasing the amount of total plasmid in vitro, where conversion of gapped plasmid in the plasmid solution to nicked plasmid is not considered increasing the amount of total plasmid. In some embodiments, the enzymatic conversion reactions may be performed so that the total amount of plasmid is substantially unchanged. In other embodiments, some or substantial plasmid may be lost, such as potentially in the optional exonuclease reaction.

Preferably, the enzymatic conversion reactions are performed so that the amount of supercoiled plasmid is increased from the amount of supercoiled plasmid immediately prior to the enzymatic conversion reactions. Preferably, this is accomplished without increasing in vitro the number of plasmid molecules.

Preferably, the enzymatic conversion reactions are performed so that the percentage of supercoiled plasmid is increased from the percentage of supercoiled plasmid immediately prior to the enzymatic conversion reactions. Preferably, this is accomplished without separation of open circular plasmid from supercoiled plasmid prior to or during the second reaction.

Preferably, the enzymatic conversion reactions are performed substantially without using a strand displacing DNA polymerase, which generates displaced single stranded DNA.

Preferably, the enzymatic conversion reactions are performed in a manner to minimize or avoid in vitro recombination events. For example, the enzymatic conversion reactions are preferably performed in the absence of RecA protein or in the absence of single stranded DNA binding protein, both of which promote recombination events. Preferably, the enzymatic conversion reactions are performed without purposeful conversion of plasmid to triple stranded forms, Holliday structures, or other strand invasion forms, and without prior such in vitro conversion.

Preferably, the enzymatic conversion reactions are performed using purified enzymes. This can be accomplished by using recombinant enzymes purified by chromatography. Preferably, the enzymatic conversion reactions are not performed using a crude extract as a source of enzyme, such as a cell lysate. Preferably, the enzymatic conversion reactions will produce supercoiled plasmid which is not modified; therefore, the enzymatic conversion reactions are preferably performed in the absence of nucleotide analogs, which would otherwise introduce modified nucleotides into the supercoiled plasmid.

Preferably, the unligatable open circular plasmid employed in the enzymatic conversion reactions is derived from (i) unligatable open circular plasmid which exists in host cells immediately prior to lysis, (ii) supercoiled plasmid in host cells which is unintentionally converted to unligatable open circular plasmid in the preparation of the cleared lysate, or (iii) supercoiled plasmid in the cleared lysate which is unintentionally converted to unligatable open circular plasmid after further plasmid purification prior to the enzymatic conversion reactions. Unintentional conversion is the consequence of the inherent instability of supercoiled plasmid to DNA damage.

Preferably, prior to the enzymatic conversion reactions, open circular plasmid in the plasmid solution is not derived from an in vitro enzymatic reaction which produces open circular plasmid from non-open circular plasmid. For example, prior to the enzymatic conversion reactions, unligatable open circular plasmid is preferably not derived from single stranded circular DNA (non-plasmid), which is converted to open circular plasmid by an in vitro enzymatic reaction.

It will be appreciated that the enzymatic conversion reactions may not result in 100% yield. Unintentional plasmid modification may occur. This unintentional conversion may be the result of enzyme impurities. For example, nuclease contamination may convert supercoiled plasmid to open circular plasmid. Unintentional conversion may also result from the side reactions due to inherent activities of the enzymes used. Several examples illustrate this point. (1) Exonuclease V may convert a small amount of gapped plasmid to linear form, by hydrolysis of the single stranded region of the gapped plasmid. This conversion is not considered purposeful, since the purpose of exonuclease V is hydrolysis of chromosomal DNA. (2) DNA polymerase I may produce a tiny amount of displaced strand as a side reaction, despite the fact that it possesses 5′-3′ exonuclease activity. This is not considered purposeful strand displacement, since one purpose of DNA polymerase I is repairing open circular plasmid by nick translation. (3) An AP endonuclease (such as exonuclease III) may convert a small amount of supercoiled plasmid to nicked plasmid, if the supercoiled plasmid contains an abasic site. This is not considered purposeful nicking, since the purpose of the AP endonuclease is the repair of open circular plasmid.

### Enzyme Reagents

Performing the enzymatic conversion reactions is facilitated by using premixed enzyme reagents. A preferred enzyme composition comprises DNA polymerase, DNA ligase, and DNA gyrase. The preferred composition may further comprise one or more 3′ deblocking enzymes. The 3′ deblocking enzyme may be 3′-phosphatase, 3′-5′ exonuclease (e.g. exonuclease III), AP endonuclease (e.g. endonuclease IV), PNKP, other deblocking enzyme(s), or a combination thereof. The preferred composition may further comprise one or more 5′ deblocking enzymes. A preferred enzyme composition for the alternate mode comprises polynucleotide kinase, 3′-phosphatase, DNA ligase, and DNA gyrase.

To the inventor's knowledge, the enzyme compositions comprising 3′-phosphatase do not exist in nature. According to the prior art, DNA gyrase exists only in prokaryotes whereas 3′-phosphatase exists only in eukaryotes.

Preferably, the enzyme composition does not comprise additional enzymes which result in (i) in vitro plasmid replication and (ii) conversion of single stranded circular DNA to open circular DNA without using a synthetic primer. Examples of such additional enzymes include primase, RNA polymerase, and single stranded DNA binding protein. Preferably, the enzyme composition does not comprise topoisomerase I.

The enzyme composition may further comprise one or more of the following enzymes: (1) kinase enzyme to convert the nucleotide by-product of cofactor back to cofactor, (2) inorganic pyrophosphatase, (3) one or more exonucleases to selectively hydrolyze residual chromosomal DNA, (4) topoisomerase IV, and (5) ribonuclease to hydrolyze residual RNA contamination.

The enzymes of the composition could be produced using recombinant DNA technology as genetic fusions with affinity fusion protein tags to facilitate purification. For example, the enzymes could be fused to glutathione-S-transferase or polyhistidine, and then purified by affinity chromatography on glutathione agarose or nickel chelating resin respectively. The enzymes could be purified to decrease endotoxin contamination to low levels. The enzyme reagents could be supplied in dry lyophilized form (optionally with trehalose), as a stabilized hydrogel, or as an aqueous solution (e.g. buffered 50% glycerol solution).

### Advantages Over Prior Art

The present invention offers three potential fundamental advantages over prior art methods: (1) increased yield of supercoiled plasmid, (2) uniformly highly supercoiled state, and (3) one universal procedure for all plasmids. These advantages are discussed further.

The above methods differ in a fundamental manner from prior art methods for purifying supercoiled plasmid. Prior art methods are based on excluding open circular plasmid from the final plasmid preparation. The invention is based on including derivatives of open circular plasmid in the final plasmid preparation, by enzymatically converting open circular plasmid to supercoiled plasmid. Surprisingly and unexpectedly, using a preferred mode of the first reaction, nearly all of the open circular plasmid can be converted to supercoiled plasmid.

As a consequence of the inclusion principle, one advantage over prior art methods is increased supercoiled plasmid yield. In some embodiments, the inventor has observed substantially no loss of plasmid in the enzymatic conversion steps. This is illustrated in Examples 1 and 2. In contrast, prior art methods are based on separation, which involves loss of plasmid. In prior art methods, the open circular plasmid is lost during the separation process. In addition, some supercoiled plasmid is also lost in any prior art separation process due to imperfect resolution of separation.

For example, assume that a plasmid preparation has 25% open circular plasmid and 75% supercoiled plasmid. Using prior art methods, the theoretical maximum yield of supercoiled plasmid is 75% of the starting amount of total plasmid. Additional supercoiled plasmid is likely to be lost in the separation. Here, the theoretical maximum yield of supercoiled plasmid may be 100% of the starting amount of total plasmid. Concern about loss of supercoiled plasmid due to damage which converts it to open circular form (e.g. during the fermentation, producing cleared lysate, or further purifying the plasmid to create the plasmid solution) is eliminated, because open circular plasmid will be converted to supercoiled plasmid. This method may be especially useful for large plasmids, which tend to have a higher percentage of open circular plasmid due to the greater fragility of large plasmids. This method may be especially useful for large scale plasmid preparations, which tend to have a higher percentage of open circular plasmid compared to small scale plasmid preparations, due to longer processing times needed to prepare the plasmid solution.

In addition, the above methods provide a solution to a previously unrecognized problem in the art of plasmid preparation—the extent of supercoiling. The extent of supercoiling of plasmid can vary from batch to batch and under different host cell growth conditions. The extent of supercoiling may have an effect on the biological activity of the plasmid. For example, a plasmid preparation which has a low extent of supercoiling may be less bioactive than desired. In the prior art, it was reported that the extent of supercoiling of plasmid in bacteria is not at its thermodynamic maximum (Cullis et al., Biochemistry 31:9642-9646, 1992). This is due to the effect of topoisomerase I which relaxes supercoiled plasmid in the bacterial host. Thus, the extent of supercoiling in vivo is an equilibrium effect between DNA gyrase and topoisomerase I. Occasionally, the extent of supercoiling in a host may be far below normal. This poorly supercoiled plasmid could occur during the fermentation of host cells, possibly due to nutrient starvation, cell death, low ATP energy charge, the presence of deleterious intracellular proteins, or build up of deleterious extracellular by-products.

This previously unrecognized problem may be solved by DNA gyrase incubation in the third reaction. The DNA gyrase incubation could increase the extent of supercoiling to its maximum thermodynamic limit. The increased supercoiling of the plasmid could create a more condensed molecule with potentially greater transformability. The DNA gyrase incubation could convert all plasmid (including supercoiled plasmid from the cleared lysate) to a more uniformly highly supercoiled and condensed state. To the inventor's knowledge, the use of DNA gyrase in the art of plasmid preparation to solve this previously unrecognized problem has not been reported.

Surprisingly and unexpectedly, the inventor believes that a universal procedure in accordance with the invention can potentially work well for nearly all plasmids. Enzyme concentrations and enzyme incubation times may be the same for all plasmids, regardless of plasmid size, plasmid GC content, plasmid DNA sequence, percent supercoiled plasmid in the plasmid solution, and percent of chromosomal DNA contamination. In other words, the details of the procedure (such as enzyme concentrations and incubation time) would not need to be optimized for each individual plasmid. A single universal procedure may work well for all plasmids. In contrast, prior art methods usually require optimization for each individual plasmid, in order to maximize the separation of supercoiled from open circular plasmid, while minimizing loss of supercoiled plasmid. For example, effective chromatographic purification of supercoiled plasmid usually requires careful optimization of the gradient procedure and sample load amount for each individual plasmid.

A further advantage may be to ensure consistent and reproducible proportions of supercoiled plasmid in the final plasmid preparation, reducing batch to batch variation.

To the inventor's knowledge, DNA gyrase, DNA ligase, DNA polymerase, polynucleotide kinase, and 3′-phosphatase have never been applied in the field of plasmid purification. The use of these enzymes breaks new ground in the art of plasmid preparation.

Several different embodiments of the invention are demonstrated in the following non-limiting examples.

### Materials and Methods

T4 DNA ligase and human PNKP were produced as fusion proteins with glutathione-S-transferase (GST) affinity tag as follows. The genes coding for these enzymes were amplified by the polymerase chain reaction. The genes were cloned into pGEX, a commercially available expression vector (Amersham) so that the GST affinity tag was fused to the amino terminus of the enzyme. The fusion proteins were purified on glutathione-agarose according to the manufacturer's instructions. These fusion proteins are denoted GST-T4 DNA ligase and GST-PNKP. E. coli DNA gyrase was obtained from John Innes Ltd. E. coli DNA polymerase I, phage T4 DNA polymerase, phage lambda exonuclease, phage T7 exonuclease (gene 6), E. coli exonuclease I, and E. coli exonuclease III were obtained from New England Biolabs. E. coli endonuclease IV was obtained from Epicentre. M. luteus exonuclease V was obtained from USB Corp. Enzyme concentrations were not necessarily optimized in the following examples. For instance, the first part of Example 1 was repeated using one-tenth the amount of GST-T4 DNA ligase with substantially the same result.

A four kilobase plasmid in an E. coli host was prepared using the alkaline lysis method, followed by further purification to remove RNA and protein. Agarose gel electrophoresis showed approximately 30% open circular plasmid, 70% supercoiled plasmid, and some residual chromosomal DNA was likely present. This plasmid preparation, denoted p4kb, was used in the subsequent examples. A 10-kilobase plasmid, denoted p10kb, was prepared in the same manner, comprising approximately 50% open circular plasmid and 50% supercoiled plasmid.

### EXAMPLE 1

### Preferred Mode

A 10 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase, 2.8 μg GST-T4 DNA ligase, 0.2 units DNA polymerase I, 200 μM dATP, 200 μM dGTP, 200 μM dCTP, and 200 μM dTTP. This reaction was incubated at 37° C. for 2 hours. After incubation, the plasmid was analyzed by agarose gel electrophoresis. The gel showed a high yield of supercoiled plasmid, confirming conversion of most of the open circular plasmid to supercoiled plasmid. By visual inspection of the stained gel, it is estimated that about 80% to 85% of open circular plasmid was converted to supercoiled form. Based on flourometry analysis, the total amount of plasmid measured before and after the reaction was the same. Extending the incubation time to 4 hours resulted in about 95% conversion. A 2-hour incubation using 1 μg p4kb resulted in about 95% conversion. A 4-hour incubation using 15 μg p4kb resulted in about 85% conversion. A 2-hour incubation using 5 μg p10kb resulted in about 80-85% conversion. A 2-hour incubation using 5 μg p4kb and 0.2 units T4 DNA polymerase (instead of DNA polymerase I) resulted in about 40% conversion. As a control, a 2-hour incubation using 5 μg p4kb and only the enzymes GST-T4 DNA ligase and DNA gyrase resulted in only about 5% conversion.

### EXAMPLE 2

### Preferred Mode+3′ Deblocking Enzyme

A 10 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase, 2.8 μg GST-T4 DNA ligase, 0.2 units DNA polymerase 1,200 μM dATP, 200 μM dGTP, 200 μM dCTP, 200 μM dTTP, and 0.5 units exonuclease III. This reaction was incubated at 37° C. for 2 hours. After incubation, the plasmid was analyzed by agarose gel electrophoresis. The gel showed high purity supercoiled plasmid, confirming conversion of virtually all of the open circular plasmid to supercoiled plasmid. The open circular band was barely visible on the gel. By visual inspection of the stained gel, it is estimated that greater than about 95% to 99% of open circular plasmid was converted to supercoiled form. Based on flourometry, the total amount of plasmid measured before and after the reaction was the same. A 4-hour incubation using 15 μg p4kb resulted in greater than 95% conversion. A 2-hour incubation using 5 μg p10kb resulted in about 95% conversion. A 2-hour incubation using 5 μg p4kb and 1 unit endonuclease IV (instead of exonuclease III) resulted in greater than about 95% to 99% conversion. A 2-hour incubation using 5 μg p4kb and 1.4 μg GST-PNKP (instead of exonuclease III) resulted in about 90% to 95% conversion.

### EXAMPLE 3

### Preferred Mode+ATP Regeneration

A 10 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase, 2.8 μg GST-T4 DNA ligase, 0.2 units DNA polymerase 1,200 μM dATP, 200 μM dGTP, 200 μM dCTP, 200 μM dTTP, 0.05 units creatine kinase (Sigma C3755), and 1 mM creatine phosphate. This reaction was incubated at 37° C. for 2 hours. After incubation, the plasmid was analyzed by agarose gel electrophoresis. The gel showed high purity supercoiled plasmid, confirming conversion of most of the open circular plasmid to supercoiled plasmid. By visual inspection of the stained gel, it is estimated that about 75% to 80% of open circular plasmid was converted to supercoiled form.

### EXAMPLE 4

### Preferred Mode+Concurrent Exonuclease Digestion

A 10 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 2.5 units DNA gyrase, 2.8 μg GST-T4 DNA ligase, 0.2 units DNA polymerase 1,200 μM dATP, 200 μM dGTP, 200 μM dCTP, 200 μM dTTP, and 0.5 units exonuclease V. This reaction was incubated at 37° C. for 2 hours. After the incubation, the plasmid was analyzed by agarose gel electrophoresis. The gel showed high purity supercoiled plasmid, confirming conversion of most of the open circular plasmid to supercoiled plasmid. By visual inspection of the stained gel, it is estimated that about 80% to 85% of open circular plasmid was converted to supercoiled form.

A 2-hour incubation using 1 unit lambda exonuclease and 5 units exonuclease I (instead of exonuclease V) resulted in about 80% to 85% conversion. Based on flourometry, in the latter experiment, the loss of DNA in the enzymatic reaction was about 3%. This DNA loss is likely a loss of some chromosomal DNA and possibly a loss of a small amount of plasmid. In separate experiments, the inventor has determined that lambda exonuclease is able to degrade a small amount of open circular plasmid, due to lack of absolute substrate specificity for linear DNA. Based on the stained agarose gel, this plasmid preparation contained slightly less open circular plasmid than the same incubation performed without the exonucleases.

### EXAMPLE 5

### Preferred Mode+Subsequent Exonuclease Digestion

A 20 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 2.5 units DNA gyrase, 2.8 μg GST-T4 DNA ligase, 0.2 units DNA polymerase I, 200 μM dATP, 200 μM dGTP, 200 μM dCTP, and 200 μM dTTP. This reaction was incubated at 37° C. for 2 hours. After this enzymatic conversion reaction incubation, the following exonucleases were subsequently added: 0.5 μl 20 units/μl exonuclease I, 1.0 μl 10 units/μl T7 exonuclease, and 1.0 μl 10 units/μl exonuclease III. The reaction was incubated an additional 2 hours at 37° C. After incubation, the plasmid was analyzed by agarose gel electrophoresis. The stained gel showed only supercoiled plasmid, with no visible open circular plasmid. Based on flourometry, the loss of DNA in the subsequent exonuclease incubation was about 12%. This DNA loss is likely a loss of both linear chromosomal DNA and residual open circular plasmid. This residual open circular plasmid, remaining after the enzymatic conversion reactions, is subsequently degraded by both exonuclease III and T7 exonuclease. Separate experiments by the inventor suggest that this exonuclease mixture, used at this concentration and duration, may reduce linear chromosomal DNA contamination by 50 fold. Based on visual inspection of the stained gel, no significant degradation of supercoiled plasmid was observed by this subsequent exonuclease incubation.

### EXAMPLE 6

### Alternate Mode

A 10 μl reaction volume contained 5 μg p4kb plasmid, 35 mM Tris-HCl (pH 7.5), 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase, 2.8 μg GST-T4 DNA ligase, and 1.4 μg GST-PNKP. This reaction was incubated at 37° C. for 2 hours. After incubation, the plasmid was analyzed by agarose gel electrophoresis. The gel showed conversion of a small amount of the open circular plasmid to supercoiled plasmid. Conversion of some open circular plasmid to supercoiled form was confirmed using purified open circular p4kb. Based on flourometry, the total amount of plasmid measured before and after the reaction was the same.

Patents, patent applications, books, and other publications cited herein are incorporated by reference in their entirety.

All modifications and substitutions that come within the meaning of the claims and the range of their legal equivalents are to be embraced within their scope.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific prior art embodiments that would anticipate the claimed invention or destroy novelty.

Moreover, no particular relationship between or among limitations of a claim is intended unless such relationship is explicitly recited in the claim (e.g. the arrangement of components in a product claim or order of reactions in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method for preparing plasmid from host cells, wherein the host cells contain the plasmid, the method comprising:
(a) providing a plasmid solution comprising unligatable open circular plasmid;
(b) reacting in vitro the unligatable open circular plasmid with one or more enzymes and appropriate nucleotide cofactors, such that at least some unligatable open circular plasmid is converted to 3′-hydroxyl, 5′-phosphate nicked plasmid;
(c) reacting in vitro the 3′-hydroxyl, 5′-phosphate nicked plasmid with a DNA ligase and DNA ligase nucleotide cofactor, such that at least some 3′-hydroxyl, 5′-phosphate nicked plasmid is converted to relaxed covalently closed circular plasmid; and
(d) reacting in vitro the relaxed covalently closed circular plasmid with a DNA gyrase and DNA gyrase nucleotide cofactor, such that at least some relaxed covalently closed circular plasmid is converted to negatively supercoiled plasmid.

2. The method according to claim 1, wherein reaction (b) is performed with a DNA polymerase in the presence of deoxyribonucleoside triphosphates.

3. The method according to claim 2, wherein reaction (b) is performed with
(i) a DNA polymerase having both 3′-5 and 5′-3 exonuclease activities; or
(ii) a DNA polymerase lacking 3′-5′ exonuclease activity and having 5′-3′ exonuclease activity, and a 3′ deblocking enzyme; or
(iii) a DNA polymerase having 3′-5′ exonuclease activity and lacking 5′-3′ exonuclease activity, and a 5′ deblocking enzyme; or
(iv) a DNA polymerase lacking both 3′-5′ and 5′-3′ exonuclease activities, a 3′ deblocking enzyme, and a 5′ deblocking enzyme.

4. The method according to claim 2, wherein reaction (b) is performed with a DNA polymerase, and optionally a 3′ debocking enzyme, and optionally a 5′ deblocking enzyme, and wherein at least one repair activity is provided for the 3′ terminus of open circular plasmid and at least one repair activity is provided for the 5′ terminus of open circular plasmid.

5. The method according to claim 2, wherein the DNA polymerase is DNA polymerase 1, having both 3′-5′ and 5′-3′ exonuclease activities.

6. The method according to claim 2, wherein reactions (b), (c), and (d) are combined in a single in vitro incubation, by incubating with a mixture comprising a DNA polymerase, DNA ligase, and DNA gyrase.

7. The method according to claim 6, wherein the mixture further comprises a kinase enzyme, wherein said kinase enzyme converts the nucleotide by-product of DNA gyrase nucleotide cofactor back to nucleotide cofactor in the presence of a high energy phosphate donor.

8. The method according to claim 6, wherein the plasmid solution further comprises linear chromosomal DNA and the mixture further comprises one or more exonuclease(s), wherein the exonuclease(s) selectively degrade the linear chromosomal DNA without substantially degrading open circular plasmid, relaxed covalently closed circular plasmid, and supercoiled plasmid.

9. The method according to claim 6, wherein the plasmid solution further comprises linear chromosomal DNA and the mixture further comprises one or more exonuclease(s), wherein the exonuclease(s) have at least some substrate selectivity in preferentially degrading linear chromosomal DNA substrate versus open circular and covalently closed circular plasmid substrates, whereby at least some linear chromosomal DNA is degraded.

10. The method according to claim 1, wherein reaction (b) is performed with a 3′ deblocking enzyme, DNA polymerase, and deoxyribonucleoside triphosphates.

11. The method according to claim 10, wherein reactions (b), (c), and (d) are combined in a single in vitro incubation, by incubating with a mixture comprising a 3′ deblocking enzyme, DNA polymerase, DNA ligase, and DNA gyrase.

12. The method according to claim 11, wherein the plasmid solution further comprises linear chromosomal DNA and the mixture further comprises one or more exonuclease(s), wherein the exonuclease(s) selectively degrade the linear chromosomal DNA without substantially degrading open circular plasmid, relaxed covalently closed circular plasmid, and supercoiled plasmid.

13. The method according to claim 11, wherein the plasmid solution further comprises linear chromosomal DNA and the mixture further comprises one or more exonuclease(s), wherein the exonuclease(s) have at least some substrate selectivity in preferentially degrading linear chromosomal DNA substrate versus open circular and covalently closed circular plasmid substrates, whereby at least some linear chromosomal DNA is degraded.

14. The method according to claim 10, wherein the 3′ deblocking enzyme is selected from the group consisting of 3′-5′ exonuclease, apurinic/apyrimidinic endonuclease, phosphatase, 3′-phosphodiesterase, and combinations thereof.

15. The method according to claim 1, wherein the plasmid solution further comprises linear chromosomal DNA and the method further comprises (e) reacting in vitro the linear chromosomal DNA with one or more exonuclease(s), wherein said exonuclease(s) selectively degrade the linear chromosomal DNA without substantially degrading relaxed covalently closed circular plasmid and supercoiled plasmid.

16. The method according to claim 1, wherein the plasmid solution further comprises linear chromosomal DNA and the method further comprises (e) reacting in vitro the linear chromosomal DNA with one or more exonuclease(s), wherein said exonuclease(s) have at least some substrate selectivity in preferentially degrading linear chromosomal DNA substrate versus covalently closed circular plasmid substrate, whereby at least some linear chromosomal DNA is degraded.

17. The method according to claim 1, wherein the plasmid solution further comprises linear chromosomal DNA and the method further comprises (e) reacting in vitro the linear chromosomal DNA with one or more exonuclease(s) prior to completing reaction (c), wherein said exonuclease(s) have at least some substrate selectivity in preferentially degrading linear chromosomal DNA substrate versus open circular and covalently closed circular plasmid substrates, whereby at least some linear chromosomal DNA is degraded.

18. The method according to claim 15, wherein the exonuclease(s) is selected from the group consisting of exonuclease 1, exonuclease III, exonuclease V, exonuclease VII, exonuclease VIII, lambda exonuclease, T5 exonuclease, T7 exonuclease, and combinations thereof.

19. The method according to claim 1, wherein reaction (d) results in less than 20% of total plasmid in catenated form.

20. The method according to claim 1, wherein greater than 75% of open circular plasmid in the plasmid solution is converted to supercoiled plasmid by reactions (b), (c), and (d).

21. The method according to claim 1, wherein reaction (b) is performed with 3′-phosphatase and polynucleotide kinase.

22. The method according to claim 21, wherein reactions (b), (c), and (d) are combined in a single in vitro incubation by reacting with a mixture comprising polynucleotide kinase, 3′-phosphatase, DNA ligase, and DNA gyrase.

23. The method according to claim 1, wherein the plasmid solution is produced by preparing a cleared lysate of the host cells.

24. The method according to claim 1, wherein the plasmid solution is produced by preparing a cleared lysate of the host cells and further purifying plasmid from other host cell components.

25. The method according to claim 24, wherein unligatable open circular plasmid in the plasmid solution consists essentially of (i) unligatable open circular plasmid which was present in the host cells prior to cell lysis, (ii) supercoiled plasmid in the host cells which was unintentionally converted to unligatable open circular plasmid during preparation of the cleared lysate, (iii) supercoiled plasmid in the cleared lysate which was unintentionally converted to unligatable open circular plasmid by further purification of plasmid from other host cell components, or (iv) a combination thereof.

26. The method according to claim 1, wherein the plasmid solution further comprises supercoiled plasmid and reactions (b), (c), and (d) are performed (i) without prior purposeful conversion of the supercoiled plasmid to linear form, (ii) without prior purposeful conversion of supercoiled plasmid to open circular plasmid, (iii) without prior purposeful conversion of supercoiled plasmid to relaxed covalently closed circular plasmid, and (iv) without prior purposeful conversion of open circular plasmid of (a) to single stranded circular DNA.

27. The method according to claim 1, wherein reactions (b), (c), and (d) are performed without in vitro plasmid replication and without prior in vitro plasmid replication.

28. The method according to claim 1 further comprising preparing a cleared lysate of the host cells, wherein the cleared lysate comprises the unligatable open circular plasmid.

29. The method according to claim 1, wherein the unligatable open circular plasmid was synthesized by the host cells.

30. The method according to claim 1, wherein the plasmid solution does not comprise purposefully in vitro synthesized, open circular plasmid.

31. The method according to claim 1, wherein the plasmid solution further comprises supercoiled plasmid and reactions (b), (c), and (d) are performed without purposeful in vitro conversion and without prior purposeful in vitro conversion of the supercoiled plasmid to an undesired form; and wherein reactions (b) and (c) are performed without purposeful in vitro conversion and without prior purposeful in vitro conversion of the open circular plasmid to an undesired form.

32. The method according to claim 1, wherein the plasmid solution further comprises supercoiled plasmid and reactions (b) and (c) are performed without prior purposeful separation of open circular plasmid from supercoiled plasmid.

33. The method according to claim 1, wherein reactions (b), (c), and (d) are performed so that the total amount of plasmid is substantially unchanged.

34. The method according to claim 1 further comprising recovering the supercoiled plasmid after reaction (d).

35. The method according to claim 35 further comprising transforming the recovered plasmid into recipient cells.

36. A method for preparing plasmid from host cells, wherein the host cells contain the plasmid, the method comprising:
(a) providing a plasmid solution comprising unligatable open circular plasmid;
(b) reacting in vitro the unligatable open circular plasmid with one or more enzymes and appropriate nucleotide cofactors, such that at least some unligatable open circular plasmid is converted to 3′-hydroxyl, 5′-phosphate nicked plasmid;
(c) reacting in vitro the 3′-hydroxyl, 5′-phosphate nicked plasmid with a DNA ligase in the presence of DNA ligase nucleotide cofactor, such that at least some 3′-hydroxyl, 5′-phosphate nicked plasmid is converted to relaxed covalently closed circular plasmid; and
(d) reacting in vitro the relaxed covalently closed circular plasmid with a reverse DNA gyrase and reverse DNA gyrase nucleotide cofactor, such that at least some relaxed covalently closed circular plasmid is converted to positively supercoiled plasmid.

37. A method for preparing plasmid from host cells, wherein the host cells contain the plasmid, the method comprising:
(a) providing a plasmid solution comprising unligatable open circular plasmid and supercoiled plasmid;
(b) reacting in vitro the unligatable open circular plasmid with one or more enzymes and appropriate nucleotide cofactors, such that at least some unligatable open circular plasmid is converted to 3′-hydroxyl, 5′-phosphate nicked plasmid;
(c) reacting in vitro the 3′-hydroxyl, 5′-phosphate nicked plasmid with a DNA ligase and DNA ligase nucleotide cofactor, such that at least some 3′-hydroxyl, 5′-phosphate nicked plasmid is converted to relaxed covalently closed circular plasmid, wherein the relaxed covalently closed circular plasmid is not further converted enzymatically in vitro to supercoiled plasmid; and
(d) recovering the supercoiled plasmid and the relaxed covalently closed circular plasmid;
wherein reactions (b) and (c) are performed without purposeful in vitro conversion and without prior purposeful in vitro conversion of (i) the supercoiled plasmid to an undesired form, and (ii) open circular plasmid to an undesired form; and
wherein reactions (b) and (c) are performed without prior purposeful separation of unligatable open circular plasmid from supercoiled plasmid.

38. The method according to claim 37, wherein the plasmid solution further comprises linear chromosomal DNA and the method further comprises (e) reacting in vitro the linear chromosomal DNA with one or more exonuclease(s), wherein said exonuclease(s) selectively degrade the linear chromosomal DNA without substantially degrading relaxed covalently closed circular plasmid and supercoiled plasmid.

39. The method according to claim 37, wherein the plasmid solution further comprises linear chromosomal DNA and the method further comprises (e) reacting in vitro the linear chromosomal DNA with one or more exonuclease(s), wherein said exonuclease(s) have at least some substrate selectivity in preferentially degrading linear chromosomal DNA substrate versus covalently closed circular plasmid substrate, whereby at least some linear chromosomal DNA is degraded.

40. The method according to claim 37 further comprising transforming the recovered plasmid into recipient cells.

41. An enzyme composition useful for converting unligatable open circular plasmid to supercoiled plasmid comprising: 3′ deblocking enzyme, DNA polymerase, DNA ligase, and DNA gyrase.

42. The composition according to claim 41 further comprising a kinase enzyme, wherein said kinase enzyme converts the nucleotide by-product of DNA gyrase nucleotide cofactor back to nucleotide cofactor in the presence of a high energy phosphate donor.

43. The composition according to claim 41 further comprising one or more exonuclease(s), wherein the exonuclease(s) selectively degrades linear chromosomal DNA without substantially degrading open circular plasmid, relaxed covalently closed circular plasmid, and supercoiled plasmid.

44. The composition according to claim 41 further comprising one or more exonuclease(s), wherein the exonuclease(s) have at least some substrate selectivity in preferentially degrading linear chromosomal DNA substrate versus open circular and covalently closed circular plasmid substrates.

45. The composition according to claim 41, wherein the 3′ deblocking enzyme is selected from the group consisting of 3′-5′ exonuclease, apurinic/apyrimidinic endonuclease, phosphatase, 3′-phosphodiesterase, and combinations thereof.

46. An enzyme composition useful for converting unligatable open circular plasmid to supercoiled plasmid comprising: DNA polymerase, DNA ligase, DNA gyrase, and one or more exonuclease(s); wherein the exonuclease(s) selectively degrades linear chromosomal DNA without substantially degrading open circular plasmid, relaxed covalently closed circular plasmid and supercoiled plasmid.

47. An enzyme composition useful for converting unligatable open circular plasmid to supercoiled plasmid comprising: DNA polymerase, DNA ligase, DNA gyrase, and one or more exonuclease(s); wherein the exonuclease(s) have at least some substrate selectivity in preferentially degrading linear chromosomal DNA substrate versus open circular and covalently closed circular plasmid substrates.

48. An enzyme composition useful for converting unligatable open circular plasmid to supercoiled plasmid comprising: DNA gyrase, DNA ligase, polynucleotide kinase, and 3′-phosphatase.

49. A kit for converting unligatable open circular plasmid to supercoiled plasmid comprising: (a) DNA polymerase, (b) DNA ligase, (c) DNA gyrase or reverse DNA gyrase, and (d) 3′-deblocking enzyme and/or exonuclease in one or more containers.

50. A kit for converting unligatable open circular plasmid to supercoiled plasmid comprising: (a) DNA polymerase, (b) DNA ligase, (c) DNA gyrase, and (d) instructions to practice the method according to claim 1.

51. A kit for converting unligatable open circular plasmid to supercoiled plasmid comprising: (a) DNA polymerase, (b) DNA ligase, and (c) instructions to practice the method according to claim 37.
